(19) United States
(12) Patent Application Publication
(10) Pub. No.: US 2023/0013814 A1
(43) Pub. Date: Jan. 19, 2023

(54) SYSTEM AND METHOD FOR AUTOMATED ENDURANCE TESTING

(71) Applicant: Chirp Inc., Waterloo (CA)

(72) Inventor: Parthipan SIVA, Waterloo (CA)

(21) Appl. No.: 17/846,575

(22) Filed: Jun. 22, 2022

Related U.S. Application Data
(60) Provisional application No. 63/221,065, filed on Jul. 13, 2021.
(60) Provisional application No. 63/221,074, filed on Jul. 13, 2021.

(51) Int. Cl. A61B 5/11; A61B 6/00; A61B 5/00

(52) U.S. Cl. CPC A61B 5/11; A61B 6/541; A61B 5/4869

(57) ABSTRACT

A system for endurance testing including: at least one sensor configured to collect data associated with an individual's movement for an endurance test; an action module configured to determine endurance movements from the collected data and determine a set of test action; and an analysis module configured to analyze the set of test actions to provide a result for the endurance test. A method for endurance testing including: collecting data associated with an individual's movement, via at least one sensor; determining endurance movements from the collected data; determining a set of test action from the endurance movements; analyzing the set of test actions; and providing results associated with the endurance test based on the analyzed set of test actions.

### RELATED APPLCIATIONS

The present disclosure claims the benefit of U.S. Provisional Application No. 63/221,065, filed Jul. 13, 2021, and of U.S. Provisional Application No. 63/221,074, filed July 13, 2021, both of which are incorporated herein in their entirety.

### FIELD

The present disclosure relates generally to a system and method for automated endurance testing. More particularly, the present disclosure relates to a system and method for automated passive strength and endurance testing, and in particular, for sit and stand testing using privacy preserving sensors.

### BACKGROUND

Determining and measuring lower body strength and endurance is frequently used as an assessment tool for various situations, for example, rehabilitation and fall risk assessment. One of the tests of lower body strength is to rise from a seated position, which is generally viewed as prerequisite for functional independence. Several different types of tests are available to assess the lower body strength and endurance of an individual. A common test is the Sit to Stand test. Originally the test was designed as five times Sit to Stand test where an individual is timed to see how long it takes them to stand up from a chair 5 times with arms folded across their chest. Different timed cutoffs based on age groups have become standard and are used to flag lower body strength issues, for example: 11.4 sec for 60-69 year old, 12.6 sec for 70-79 year old and 14.8 sec for 80-89 year old.

More recently, this test has been modified to the 30 second sit to stand test (sometimes referred to as: 30 sec chair stand test). Where the number of times a person can, starting from seated position, stand and sit in a 30 second window is counted. As before the person will have their hands across the chest for unassisted sit to stand. Different cutoffs based on age and gender are provided ranging from, for example, 4 for female aged 90-94 to 14 for males aged 60-64.

There has also been proposed a modified 30 second sit to stand test to allow testing of more frail individuals. The modification is intended to allow individuals the use of their hands on the chair armrest for assistance during sit down and stand-up phase. However, once in a standing position test subject must release their hands from the armrest of the chair before continuing to sit down phase (with or without aid of the armrest).

These tests can present difficulties for automated operation and/or maintaining privacy during the testing process. As such, there is a need for an improved home strength and endurance test system and method that is privacy preserving and can be administered easily.

### SUMMARY

According to an aspect herein, there is provided a system for endurance testing including: at least one sensor configured to collect data associated with an individual's movement for an endurance test; an action module configured to determine endurance movements from the collected data and determine a set of test action; and an analysis module configured to analyze the set of test actions to provide a result for the endurance test.

In some cases, the at least one sensor may be a privacy preserving sensor.

In some cases, the at least one sensor may be an mm-wave radar sensor, a WI-FI sensor or a LIDAR sensor.

In some cases, the system may further include a trend module configured to analyze a plurality of results for the endurance test and determine the individual's endurance trends based on the plurality of results.

In some cases, the at least one sensor may be configured to notify the individual at predetermined intervals to complete an endurance test.

In some cases, the endurance test may be a predetermined time of repeating a sit to stand motion.

In some cases, the action module may be configured to perform temporal smoothing analysis on the set of test actions.

In some cases, the action module may be configured to perform radar signal processing to produce a 3D point cloud to determine actions of the individual.

In some cases, the trend module may be configured to monitor for similar action as a partial test or in an individual's daily movement to determine changes in an individual's endurance.

In some cases, the trend module may be configured to monitor for a predetermined time interval to determine changes in an individual's endurance.

In another aspect, there is provided a method for endurance testing including: collecting data associated with an individual's movement, via at least one sensor; determining endurance movements from the collected data; determining a set of test action from the endurance movements; analyzing the set of test actions; and providing results associated with the endurance test based on the analyzed set of test actions.

In some cases, the at least one sensor may be a privacy preserving sensor.

In some cases, the at least one sensor may be an mm-wave radar sensor, a WI-FI sensor or a LIDAR sensor.

In some cases, the method may further include, analyzing a plurality of results for the endurance test; and determining the individual's endurance trends based on the plurality of results.

In some cases, the method may further include, notify the individual at predetermined intervals to complete an endurance test.

In some cases, the endurance test may be a predetermined time of repeating a sit to stand motion.

In some cases, the method may further include performing temporal smoothing analysis on the set of test actions.

In some cases, the method may further include performing radar signal processing to produce a 3D point cloud to determine actions of the individual.

In some cases, the method may further include monitoring for similar action as a partial test or in an individual's daily movement to determine changes in an individual's endurance.

In some cases, the method may further include monitoring for a predetermined time interval to determine changes in an individual's endurance.

Other aspects and features of the embodiments of the system and method will become apparent to those ordinarily skilled in the art upon review of the following description of specific embodiments in conjunction with the accompanying figures.

### BRIEF DESCRIPTION OF FIGURES

Embodiments of the system and method will now be described, by way of example only, with reference to the attached Figures, wherein:

FIG. 1 illustrates an environment having a system for automating sit to stand test, according to an embodiment;

FIG. 2 is a flowchart of a method of performing an endurance (sit to stand) test, according to an embodiment;

FIG. 3 is a flowchart of a method of performing an endurance (sit to stand) test according to another embodiment;

FIG. 4 is system for automated endurance testing according to an embodiment;

FIG. 5 is a flowchart of a method of performing and analyzing an automated endurance (sit to stand) test, according to an embodiment;

FIG. 6 illustrates a method for analyzing radar signals, according to an embodiment; and

FIG. 7 is a flowchart of a method of determining actions based on an endurance (sit to stand) test according to an embodiment.

### DETAILED DESCRPTION

In the following, various example systems and methods will be described herein to provide example embodiment(s). It will be understood that no embodiment described below is intended to limit any claimed invention. The claims are not limited to systems, apparatuses or methods having all of the features of any one embodiment or to features common to multiple or all of the embodiments described herein. A claim may include features taken from any embodiment as would be understood by one of skill in the art. The applicants, inventors or owners reserve all rights that they may have in any invention disclosed herein, for example the right to claim such an invention in a continuing or divisional application and do not intend to abandon, disclaim or dedicate to the public any such invention by its disclosure in this document.

Generally, the present disclosure provides a system and method for automated endurance testing. The system and method are configured to review a user's action via at least one sensor. The system and method may review the actions performed by the user in order to determine the user's endurance testing results. The system may further note trends or anomalies with the testing.

Generally, automated testing has been considered to fall within three categories: wearables, instrumented equipment, and cameras. Wearable sensors, such as smartphones, smartwatches, or other custom wearable solutions have been used to detect and measure sit to stand actions. These wearable solutions often require the test subject to wear a device on themselves. This requires compliance and correct attachment of the wearable device, which could be on the waist, legs, core, wrist, or the like. While these systems have different degree of accuracy and may further include extra metrics, such as movement of upper body, they require the test subject to wear a sensor.

Instrumented Equipment solutions, for example, sensors attached to a chair, ultrasound sensors, pressure sensors on the floor, or the like. While this type of solution is generally considered to be non-intrusive and privacy preserving, they require specific equipment to be used during testing. The sensors are generally required to be installed correctly on the chair or on the floor by the chair.

Further, there has been some reliance on camera-based systems to perform mobility testing. However, these solutions are generally not privacy preserving for use in homes and they often require calibration for obtaining, for example, upper body movement speed and distances.

Embodiments of the system and method disclosed herein are intended to provide for an automated system for self-administration or administration with a supervisor of a lower body strength and endurance test, and, in particular, a 30 Second Sit to Stand (30STS) test, in an individual's home or clinical setting. The 30STS test can be administrated through an interactive setting where the system provides instructions to the user in administrating the test. The 30STS can also be administrated passively without input or interaction required by the individual. In a passive setting, the system is configured to continuously monitors for any occurrence of the 30STS in the environment including any partially performed 30STS.

There have been attempts to automate the 30 second sit to stand test. Automation is intended to allow for unbiased counting and/or timing as well as to capture additional information, for example, upper body movement or the like. Embodiments of the system and method detailed herein may be used in a home setting, for continuous self-assessment or assessment with caregiver. Conventional wearable sensors require users to put a sensor on correctly, while instrumented chair requires specialized equipment to be installed on chairs, and video bases system require calibration in the home setting and video has privacy concerns. Embodiments of the present system and method are intended to address these concerns by providing an easy to install, privacy preserving, non-wearable sensor device that is capable of automatically detecting and measuring sit to stand movements, for example, the 30 seconds sit to stand test.

In embodiments herein, actions that may be measured for the automated sit to stand test may include:
Number of times a person performs the sit to stand action;
The duration of sit, sit to stand, stand and stand to sit actions; and
The amount of upper body movement and speed of upper body movement during sit to stand and stand to sit actions

It will be understood that other actions may also be measured depending on the type of testing or the information/data to be gathered.

To address the desire to have privacy preserving, non-wearable, no calibration device for automating 30STS, a privacy preserving sensor-based system is detailed herein. Embodiments of the system can be configured to automatically detect each component of the 30 second sit to stand (30STS) test (or alternatively 5 sit to stand 5STS test or modified 30 second sit to stand test (m30STS), or the like). In particular, the system can be generally configured to measure, for example:
Number of sit to stand events that occurred;
Duration of sit, sit to stand, stand and stand to sit actions; and
Upper body movement and velocities during sit to stand and stand to sit.

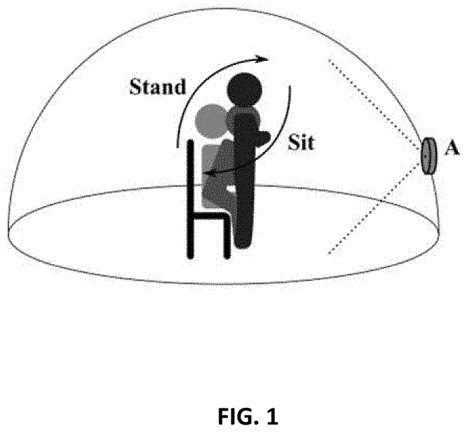

FIG. 1 illustrates an environment for a system for passive testing according to an embodiment. An ambient sensor may be attached to a wall, ceiling, or other surface within a room. It is intended that an individual perform the test or test actions within a field of view of at least one sensor. The actions of the test may be detected and measured by the system.

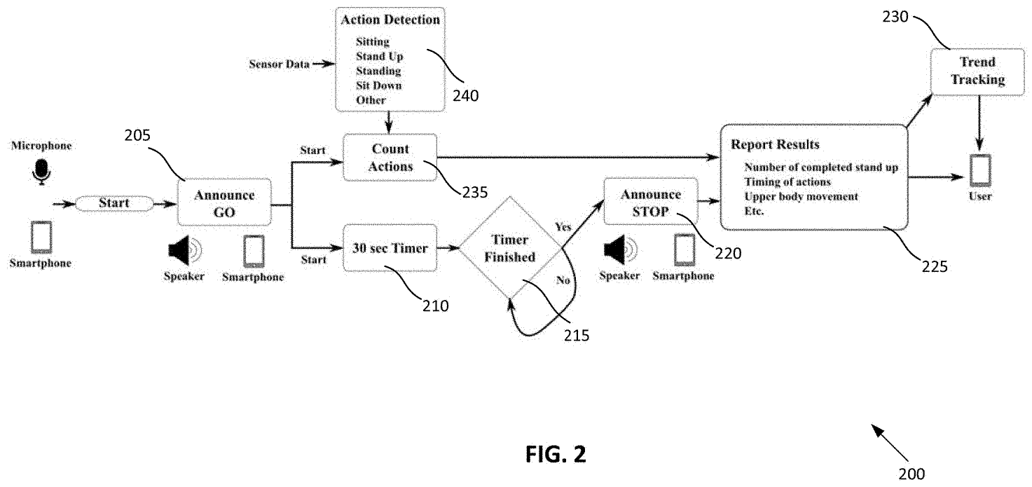

FIG. 2 illustrates a method 200 for an automated endurance (sit to stand) test. In this example, a supervisor (for example, a healthcare professional, a caregiver or the like) or the test subject themselves may setup a chair in the field of view of the sensor device (as shown in FIG. 1).

Starting in a seated position the test subject or supervisor will activate the at least one sensor for the 30STS test through, for example a voice interface or smartphone interface, at 205. The system will indicate “go” through an audible alarm at which point the test subject will repeatedly stand up and sit down as fast as safely possible when the timer is engaged at 210. At the 30 second mark, at 215 the test is completed and, at 220 the system may indicate “stop” through, for example, an audible alarm or other notification, at which point the test subject can stop. If the subject is unable to perform any further stand-up actions at a time during the testing interval, the subject may remain seated and rest until a stop is indicated by the system. After the test, the system may analyze the reports, at 225, and review and provide trend tracking at 230.

In other cases, after the system is initiated at 205 and announces the start of the test, the system may begin to count and discern the actions, at 235, as the timer is continuing. The system is configured to determine various actions, at 240, to determine if and how many times the action sequence has been completed. The system will provide results, at 225, of how many times the sit to stand action was completed and determined by the action module, within the predetermined timer interval, for example, 30 seconds.

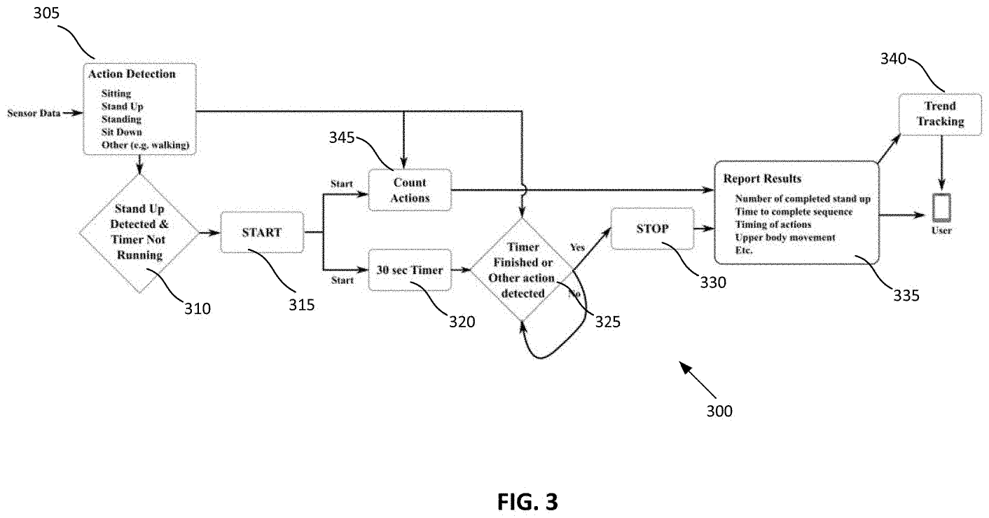

FIG. 3 illustrates a method 300 for an alternate (more passive) test for completing the 30STS test. The at least one sensor, while in passive mode, may be in a continuously armed state looking for sit to stand actions, at 305. On detection of a sit to stand action, at 310, the system may start a test at 315, to determine whether, at the same location for a 30 second, at 320, a successful 30STS test has been identified. If the timer reaches 30 or if another action is detected, at 325, the test will stop at 330, and the metrics for the 30STS may be reported, at 335. Further trend tracking may be completed after the test results have been determined, at 340.

This setting is intended for in home use where the test subject can self-administer the 30STS at any time during the day, on any chair, within the field of view of the system but could also be used in other settings. Furthermore, in the passive setting, sit to stand action (one time or more) can be timed and reported even if not repeated for the 30 second interval, at 345. Reported metrics may include, for example, a number of sit to stand observed and a duration for the entire sequence. In this way partial test results can be observed and reported.

As shown in FIG. 3, passive continuous detection of 30 sec sit to stand (30STS) test may be used in a situation where more formal testing may not be preferred. On a first occurrence of sit to stand detection, the action may trigger a 30 second timer. The 30 sec sit to stand test is ended when 30 seconds has elapsed from the start of the first sit to stand action or a different action (for example, walking) is detected. If another action is detected, before 30 seconds has elapsed, the system may register a partial completion of the 30STS test. All complete 30STS as well as partially completed 30STS tests may be reported by the system to for example, the test individual, a health care provider, or the like.

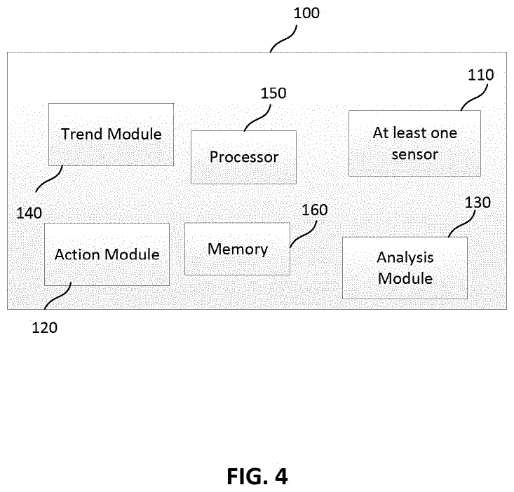

FIG. 4 illustrates a system 100 for endurance testing according to an embodiment. The system 100 includes at least one sensor 110, and action module 120, an analysis module 130, a trend module 140, at least one processor 150 and at least one memory component 150. The system is generally intended to within a device connected to a network, such as WI-FI, Bluetooth, or the like but may be distributed over various device. The modules, including the processor 150 and memory 160, are in communication with each other but may be distributed over various devices or may be housed within a single device. The processor may be configured to retrieve stored instruction from the memory 160 and execute the instructions that provide for the functionality of the modules.

The at least one sensor 110 may be used. In some cases, an mm wave radar sensor, for example a 60-64 GHz Frequency Modulated Continuous Wave (FMCW) radar having a 120 degree azimuth and elevation field of view. In some cases, the sensor may include further aspects such as a microphone, speaker, Wi-Fi component and a microprocessor. The at least one sensor may be an Mmwave radar sensor as this sensor is intended to provide for a non-wearable, privacy preserving, calibration free way of measuring people movement and detection of sit to stand actions. The microphone and speakers may be used for interactive administration of the 30STS tests and may not be needed for systems configured to passively monitor for actions. Wi-Fi is intended to be used for transferring data to and from other modules of the system and/or reports to users.

In other cases, alternative privacy preserving, calibration free sensors could also be used such as. LIDAR and WiFi sensing without altering the methodology detailed herein. LIDAR sensor are configured to provide 3D point cloud similar radar sensors but using different pre-processing methodology. In some cases, a LIDAR sensor may be more expensive than radar sensors. WiFi sensing can also be used to detect people and actions, for example using channel state information (CSI), however WiFi sensing may require computationally heavy processing to detect people and actions.

The action module 120 is configured to determine an individual's actions and determine which, if any actions collected, are 30STS actions or are other actions. The action module may time actions that are sit to stand test actions. In some cases, action that are not 30STS actions may also be reviewed. These actions may provide greater detail ad or metrics with respect to strength and stability of the individual.

The analysis module 130 is configured to review the actions and their timing determined by the action module. The analysis module is configured to provide results on 30STS tests as well as partial 30STS tests where the actions were not performed for a full 30 seconds. In some cases, the analysis module 130 may aggregate results to a full 30 second test result.

The trend module 140 is configured to review and update trends based on results determined by the analysis module 130. The trend module 140 may further notify or otherwise alert the individual, a health care provider, or the like, when actions or metrics record illustrate a stability or balance issue based on determined trends. In some cases, the trends the trend module may monitor may include, for example, consecutive decline in the number of sit and stands preformed within a 30 interval, increased upper body movement during sit to stand motions, and the like. The trend module 140 may also be configured to flag, alert, or otherwise notify a test subject, healthcare provider or the like if any results fall outside a predetermined allowable range for test subjects of similar age and gender. In some cases, the predetermined allowable range may be based on guidelines by CDC or other health agency.

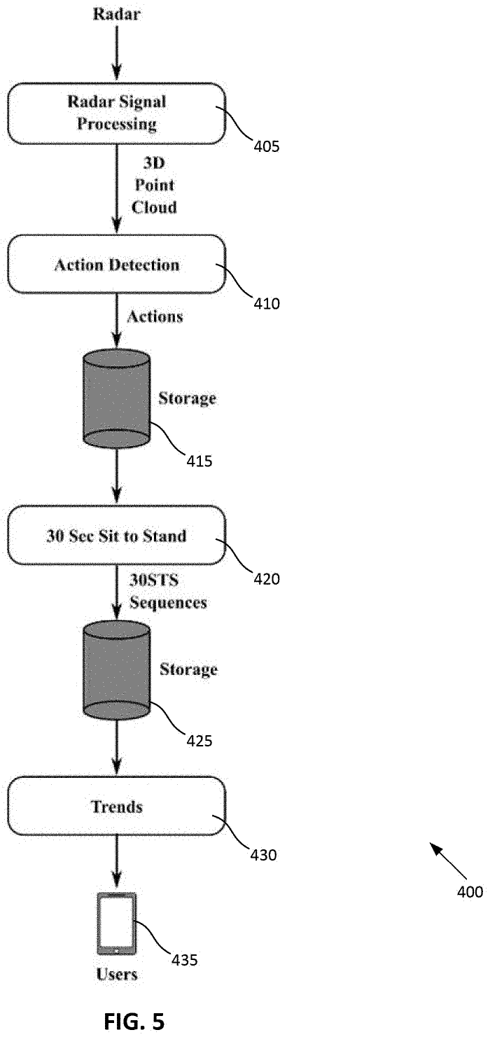

FIG. 5 illustrates a method 400 for determining results and trends from a system for passive 30STS. At 405, data is received by the system from the at least one sensor 110. In this example, a plurality of 30STS sequences may be received from at least one radar sensor to be used for trend tracking.

At 410, actions may be detected in the radar signal as it may be processed by the action module to obtain 3D point cloud of the scene. The point clouds may be stored in storage at 415 and may then be clustered and classified to detect sit and stand action, at 420. Sequences of repeated sit and stand actions at the same location may be used to detect the 30 second sit to stand test. These results may be stored at 425 and may further be used for trend tracking, at 430. At 435, the trends and results may be reported to the individual, health care professional, or the like.

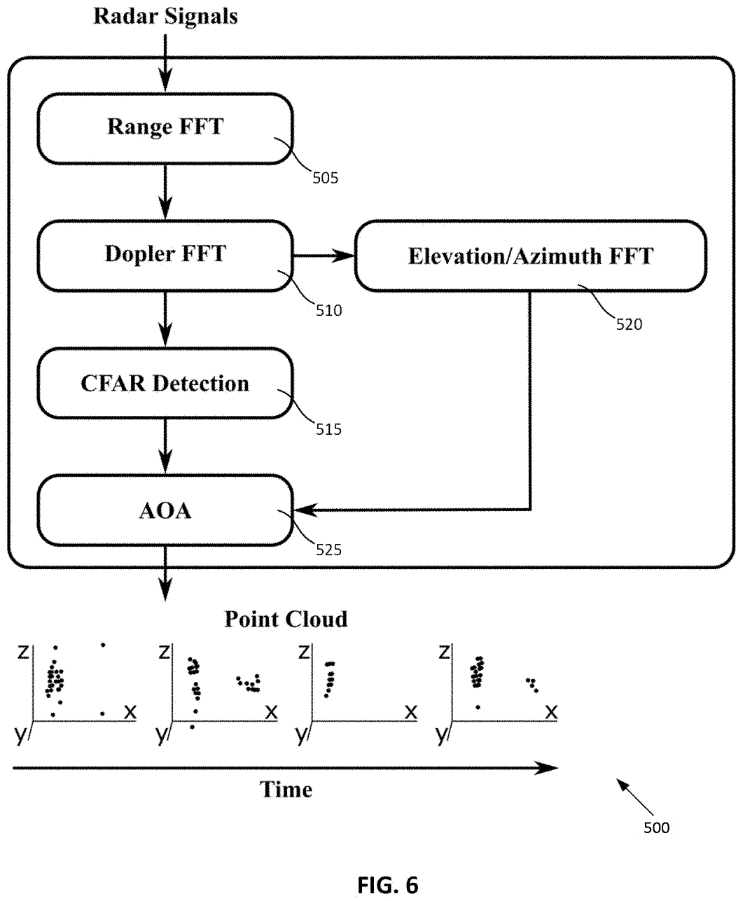

FIG. 6 illustrates a method 500 for radar signal processing according one embodiment. At 505, a range FFT and, at 510, a Doppler FFT are performed to obtain the range Doppler map. The range Doppler map may be used by the constant false alarm rate (CFAR) method, at 515 to detect objects in the scene. Using azimuth and elevation FFT, at 520, the angle of arrival (AOA) of the detected objects are determined to produce a 3D point cloud of objects in the scene, at 525. The 3D point cloud may be used for action detection. Alternatives such as the use of raw radar data, short time Fourier transforms, etc. can be used instead of the point cloud. Other pre-processing methods may be used as would be understood in the art.

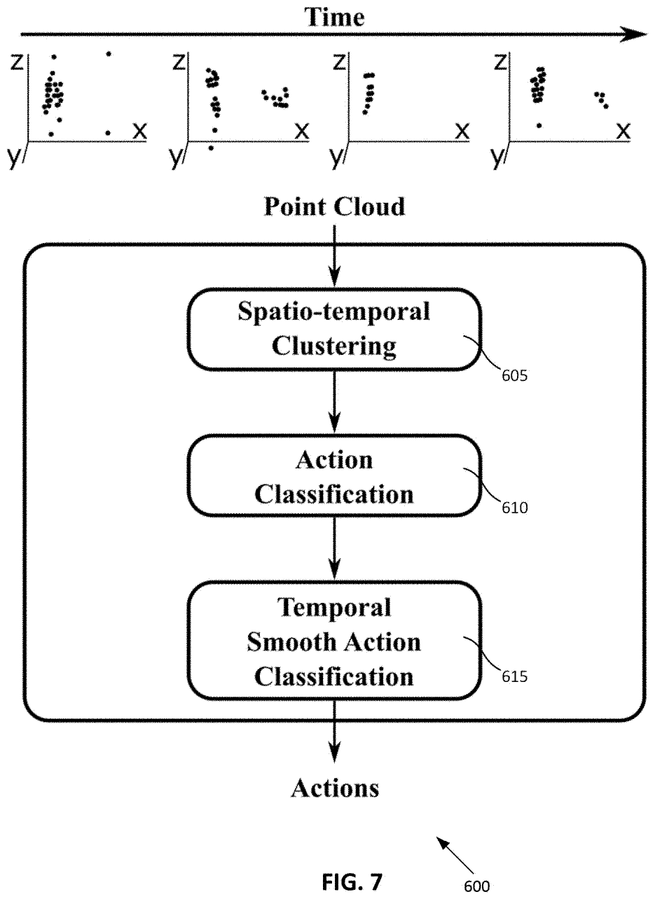

FIG. 7 illustrates a method 600 for action detection according to an embodiment. A 3D point cloud may be clustered in a space-time volume (clustering methods such as k-means or DBSCAN can be employed), at 605, and each cluster is classified as an action, at 610, (support vector machines (SVM), neural nets (NN), or other similar classifier can be used) at each time step. Temporal smoothing of action classification performed to boost accuracy of classification.

At 615, after temporal smoothing actions may be reported with a location in space and a start and end time. For the sit down and stand-up actions the movement and speeds of the upper body (as determined by the cluster movement during sit down and stand up) is identified. Temporal smoothing is intended to boost the continuous action classification accuracy. This may allow the system to better segment the transition between different actions (for example, between sit and standing up). This gives better timing of different action components.

Using the actions, identified by the action module for example via the method of FIG. 7, start and end times for the action are determined. Further, for a 30STS may be identified as a repeated detection of stand-up action in the same location in a 30 second window. These sequences are stored as the 30STS test with associated breakdown of action times and upper body movement. In an example, upper body movement may be measured as the displacement and speed of the upper body tilt at the hip. In this case, this movement may be estimated as the horizontal movement distance, speed and acceleration of the upper body during sit to stand action. It has been shown that people may start jerking their upper body to build up momentum, which is intended to help them get up while reducing strain on lower body.

For a more passive assessment, as detailed herein, each stand up action may trigger a partial 30STS test where the 30STS ends before 30 seconds if stand up action is not followed by a sit down action. In some cases, results may be reported in groups or buckets, for example, 30 sec sit stand (a full test), 25 sec sit stand (partial), 20 sec sit stand (partial), . . . , 5 sec sit stand (which may be limited to a single stand action). In some cases, the trend module may track each action of the above 5 sec to 30 sec STS separately. In some cases, the trend module may further track a total number of transfer (sit to stand) in a predetermined time period, for example, 12 hours, a day, a week or the like. By monitoring over a predetermined time period the trend module is intended to determine trends relating to the individual's endurance. For example, as subject's lower body strength starts failing, the subject (consciously or sub-consciously) may start to minimize the number of times they need to get up from a chair in a day.

In some cases, trends of 30STS tests may be reviewed over a predetermined period of time, for example, days, weeks, months years. These results may be used to form a trend and analyzed for anomalies and decline. For simplicity of understanding a linear fit to the data is used as the trend where deviation from the fit indicates anomalies and slope of the line indicates a decline. All tests falling outside the predetermined guideline, for example, the CDC guidelines, for the subject's age and gender may be flagged and an individual, health care provider or the like may be notified.

In a specific example, embodiments of the system and method detailed herein may be used by caregivers or clinicians. In some cases, embodiments of the system and method may provide for scheduled or regular endurance testing to a subject. In these cases, a subject may be alerted to commencing a test via a smartphone or other interface. The system may be configured to provide audible alerts or reminders to care recipients in order for them to complete testing at a regular or scheduled interval, for example, once a day, once a week, or the like.

Audible and scheduled reminders may further be useful as a part of physiotherapy, for example, after surgery, a fall, or the like. Providing reminders and trend analysis to a patient to perform regular testing is intended to provide the caregiver or clinician further detail as to the recovery and progression of endurance and strength of the subject.

In select embodiments, the system may further be connected to and provide reports to an occupational therapist or physiotherapist. The occupational therapist or physiotherapist may review the trends received from the system and adjust their treatment regimen (for example, exercise frequency, exercise type, or the like) for the user of the system. In select embodiments the system may also suggest particular exercise regiments to boost a users' strength and endurance on the detection of declines.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether the embodiments or elements thereof described herein are implemented as a software routine, hardware circuit, firmware, or a combination thereof.

Embodiments of the disclosure or elements thereof can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto.

What is claimed is:

1. A system for endurance testing comprising:
at least one sensor configured to collect data associated with an individual's movement for an endurance test;
an action module configured to determine endurance movements from the collected data and determine a set of test action; and
an analysis module configured to analyze the set of test actions to provide a result for the endurance test.

2. The system of claim 1, wherein the at least one sensor is a privacy preserving sensor,

3. The system of claim 2, wherein the at least one sensor is an mm-wave radar sensor, a WI-Fl sensor or a LIDAR sensor.

4. The system of claim 1 further comprising: a trend module configured to analyze a plurality of results for the endurance test and determine the individual's endurance trends based on the plurality of results.

5. The system of claim 1 wherein the at least one sensor is configured to notify the individual at predetermined intervals to complete an endurance test.

6. The system of claim 1 wherein, the endurance test is a predetermined time of repeating a sit to stand motion.

7. The system of claim 1 wherein, the action module is configured to perform temporal smoothing analysis on the set of test actions.

8. The system of claim 1 wherein, the action module is configured to perform radar signal processing to produce a 3D point cloud to determine actions of the individual.

9. The system of claim 4 wherein, the trend module is configured to monitor for similar action as a partial test or in an individual's daily movement to determine changes in an individual's endurance.

10. The system of claim 4 wherein, the trend module is configured to monitor for a predetermined time interval to determine changes in an individual's endurance.

11. A method for endurance testing comprising:
collecting data associated with an individual's movement, via at least one sensor;
determining endurance movements from the collected data;
determining a set of test action from the endurance movements;
analyzing the set of test actions; and
providing results associated with the endurance test based on the analyzed set of test actions.

12. The method of claim 11, wherein the at least one sensor is a privacy preserving sensor.

13. The method of claim 12 wherein, the at least one sensor is an mm-wave radar sensor, a WI-FI sensor or a LIDAR sensor.

14. The method of claim 11 further comprising:
analyzing a plurality of results for the endurance test; and
determining the individual's endurance trends based on the plurality of results.

15. The method of claim 14 further comprising: notify the individual at predetermined intervals to complete an endurance test.

16. The method of claim 11 wherein, the endurance test may be a predetermined time of repeating a sit to stand motion.

17. The method of claim 11 further comprising: include performing temporal smoothing analysis on the set of test actions.

18. The method of claim 11 further comprising: performing radar signal processing to produce a 3D point cloud to determine actions of the individual.

19. The method of claim 11 further comprising: monitoring for similar action as a partial test or in an individual's daily movement to determine changes in an individual's endurance.

20. The method of claim 11 further comprising: monitoring for a predetermined time interval to determine changes in an individual's endurance.
